(19) United States
(12) Patent Application Publication
(10) Pub. No.: US 2007/0185014 A1
(43) Pub. Date: Aug. 9, 2007

(54) Methods and compositions for modulating conjunctival goblet cells

(75) Inventor: Darlene A. Dartt, Newton, MA (US)

Correspondence Address: COZEN O'CONNOR, P.C., 1900 MARKET STREET, PHILADELPHIA, PA, 19103-3508, US

(73) Assignee: The Schepens Eye Research Institute, Inc., Boston, MA (US)

(21) Appl. No.: 11/351,109

(22) Filed: Feb. 9, 2006

(51) Int. Cl. A61K 38/18

(52) U.S. Cl. 514/8; 514/12

(57) ABSTRACT

The present invention relates to conjunctival goblet cell proliferation and secretions. The present invention also relates to compositions that can modulate conjunctival goblet cell proliferation and secretions. The present invention also relates to galectins, PDGF, bFGF, and the regulation of goblet cells.

### FIELD OF THE INVENTION

The present invention is related to the modulation of proliferation and secretion of conjunctival goblet cells and uses of compounds that relate to the same.

### BACKGROUND

Goblet cells are polarized epithelial cells found in columnar and stratified squamous epithelia throughout the body. They are found in the conjunctiva, nasal lacrimal duct, inner ear, intestine, colon, nasal airway, bronchial airways, and pancreatic duct. Goblet cells secrete gel-forming mucins that form the mucous layer that protects the wet-surfaced epithelia from the external environment. These cells form the first line of defense between the ocular surface, the inner ear, the gastrointestinal tract, and the respiratory tract with the external environment. Goblet cells protect these epithelium from pathogens, chemicals, mechanical trauma, dessication, and other environmental extremes. The gel forming mucins, along with other secreted molecules, are a primary mechanism of defense. The amount of mucin, as well as its proper hydration and character, is critical to the protection of the epithelial that it overlies. The amount of mucin is controlled by regulating the number of goblet cells, the rate of mucin secretion by the goblet cells, and the rate of mucin synthesis by the goblet cells.

The tear film, ubiquitously present over the surface of the eye, is composed of an overlying lipid layer, a substantial middle aqueous component, and an underlying mucous foundation. The mucous layer provides constant protection to the surface of the eye, and stability to the tear film. A rapid release of mucus in response to surface irritants, trauma, or toxins (bacterial and environmental) is necessary to replenish the mucous layer and protect the ocular surface.

Goblet cells of the conjunctiva are the primary source of mucus (complex glycoprotein) that constitutes the inner, mucous layer of the tear film. In the conjunctiva mucin secretion is stimulated by activation of a neural reflex originating from the cornea (see, for example, Dartt D A., Experimental Eye Research (2004) 74:173-185). Activation of sensory nerves in the cornea stimulate the parasympathetic and sympathetic nerves that innervate the conjunctiva and its goblet cells. The parasympathetic and sympathetic nerves release their neurotransmitters that bind to their receptors on the conjunctival goblet cells. The parasympathetic neurotransmitters acetylcholine (a cholinergic agonist) and vasoactive intestinal peptide (VIP) bind to muscarinic and VIPergic receptors to stimulate goblet cell secretion as measured in tissue (e.g. mucin, electrolytes, water, proteins, enzymes, such as peroxidases, etc). The sympathetic neurotransmitters do not stimulate goblet cell secretion.

The signaling pathway used by cholinergic agonists, e.g. acetylcholine, has been described. These agonists increase the intracellular Ca²⁺ concentration and activate calcium and protein kinase C isoforms. The protein kinase C isoforms activate the non-receptor tyrosine kinases Pyk2 and Src that in turn transactivate the epidermal growth factor receptor (EGFR). The activated EGFR, transduces its signal through various proteins (e.g. Grb2, SOS, and Ras) and activates a cascade of mitogen-activated protein kinases (MAPK) known as MAPKKK (Raf), MAPKK (MEK), and p42/p44 MAPK. The later kinase induces mucin secretion by exocytosis. It causes mucin granules stored in the goblet cell to fuse with each other and with the apical plasma membrane and subsequently release their contents onto the ocular surface. The signaling pathway used by VIP has not yet been explored.

Another G protein-linked receptor whose activation stimulates conjunctival goblet cell mucin secretion is the purinergic P2Y2 receptor. This receptor is activated by the 5′ nucleotides, ATP and UTP. These nucleotides can be released from nerves concomitantly with the neurotransmitters or from cells themselves. The signaling pathway used by these agonists has not been studied in the conjunctiva.

Gastrointestinal tract goblet cells also release mucins. Even though goblet cells in the gastrointestinal tract are not directly innervated, release of neurotransmitters from the subepithelial plexus of nerves stimulates goblet cell secretion from the intestine and colon. In the intestine electric field stimulation (induces release of neurotransmitters from all types of nerves), activation of parasympathetic nerves, and release of cholinergic agonists stimulates goblet cell mucin secretion from the crypts. This response is blunted for goblet cells in the villus tips. The neuropeptide neurotensin also stimulates goblet cell secretion. The role of VIP, also released from parasympathetic nerves, in the stimulation of secretion is controversial. Different results have been obtained depending upon the model used.

Similarly airway goblet cells release mucins. Activation of nerves increases airway mucous secretion that comes from both goblet cells and submucosal glands. Submucosal glands are present only in the cartilaginous airways, whereas goblet cells are found throughout the conducting airways. Activation of parasympathetic nerves releasing cholinergic agonists predominates in the secretory response. VIP also released by these nerves inhibits secretion. Activation of sensory nerves releasing substance P and neurokinin A also stimulates secretion, but to a lesser extent than parasympathetic nerves. Sympathetic nerves releasing norepinephrine cause a small increase in secretion. Finally purine nucleotides (ATP, UTP) cause an increase in secretion.

Neural stimulation of goblet cell secretion occurs in the three types of tissues that have been studied, but there are tissue specific differences in the type of nerve and neurotransmitter that is effective and the magnitude of the neural effect.

Regulation of normal goblet cell maturation and turnover, as well as goblet cell mucous synthesis and mucous secretion (mucous production), is important for the health of the ocular surface. In diseases and conditions such as keratoconjunctivitis sicca (KCS), Sjogren's Syndrome, vitamin A deficiency, anesthetic cornea, Stevens-Johnson Syndrome, thermal burns, chemical bums, cicatricial ocular pemphigoid, inactive trachoma, drug induced pseudopemphigoid, atopic diseases, radiation keratoconjunctivitis sicca, and superior limbic keratitis, there is an alteration in goblet cell maturation, a disruption of mucous production, and a change in the mucous layer. Other conditions and/or situations where symptoms include thin and/or unstable tear films include “dry eye” (a family of disorders including excessive tear evaporation and Non-Sjogren's aqueous tear deficiency) and contact lens related dry eye.

There is a need for the development of a means to provide a sufficient amount of mucous and/or other secretions in the eye or on the ocular surface to address the foregoing diseases and/or conditions. The present invention provides for this need as well as others.

### SUMMARY OF INVENTION

An ophthalmic composition comprising a therapeutically effective amount of galectin-1 or functional fragment thereof to treat a dry eye disorder.

An ophthalmic composition comprising a therapeutically effective amount of galectin-3 or functional fragment thereof to treat a dry eye disorder.

An ophthalmic composition comprising PDGF.

An ophthalmic composition comprising a therapeutically effective amount of bFGF or functional fragment thereof to treat a dry eye disorder.

A pharmaceutical composition comprising a therapeutically effective amount of galectin-1 or a functional fragment thereof to increase conjunctival goblet cell proliferation and/or secretion.

A pharmaceutical composition comprising a therapeutically effective amount of galectin-3 or a functional fragment thereof to increase conjunctival goblet cell proliferation and/or secretion.

A pharmaceutical composition comprising an amount of PDGF or functional fragment thereof effective to increase conjunctival goblet cell proliferation and/or secretion.

A pharmaceutical composition comprising an amount of bFGF or functional fragment thereof effective to increase conjunctival goblet cell proliferation and/or secretion.

A method of increasing conjunctival goblet cell proliferation comprising contacting a conjunctival goblet cell with a composition comprising a compound selected from the group consisting of galectin-1, galectin-3, PDGF, bFGF, or functional fragments thereof.

A method of increasing conjunctival goblet cell secretion comprising contacting a conjunctival goblet cell with a composition comprising a compound selected from the group consisting of galectin-1, galectin-3, PDGF, bFGF, or functional fragments thereof.

A method of treating dry eye in an individual comprising contacting an ocular surface of said individual with a composition comprising a compound selected from the group consisting of galectin-1, galectin-3, PDGF, bFGF, or functional fragments thereof.

A method of treating discomfort in an individual comprising a topical administration of a composition comprising a compound selected from the group consisting of galectin-1, galectin-3, PDGF, bFGF, or functional fragments thereof.

A method of increasing the age at which individuals can wear a contact lens comprising the topical administration to the ocular surface of a composition comprising a compound selected from the group consisting of galectin-1, galectin-3, PDGF, bFGF, or functional fragments thereof.

A method of stimulating goblet cell mucous secretion comprising administration to an ocular surface of a therapeutically effective amount of a preparation comprising a composition comprising a compound selected from the group consisting of galectin-1, galectin-3, PDGF, bFGF, or functional fragments thereof, further comprising infusion of the preparation from a device selected from a pump-catheter system, a selective release device, or a contact lens.

A method of stimulating goblet cell mucous secretion comprising administration to an ocular surface of a therapeutically effective amount of a preparation comprising a composition comprising a compound selected from the group consisting of galectin-1, galectin-3, PDGF, bFGF, or functional fragments thereof, wherein the preparation further comprises dispersion of said preparation in a carrier vehicle selected from the group of liquids, gels, ointments, or liposomes.

A method of stimulating goblet cell proliferation comprising administrating to an ocular surface a therapeutically effective amount of a preparation comprising a composition comprising a compound selected from the group consisting of galectin-1, galectin-3, PDGF, bFGF, or functional fragments thereof, wherein the administration further comprises infusion of the preparation from a device selected from a pump-catheter system, a selective release device, or a contact lens.

A method of stimulating goblet cell proliferation comprising administrating to an ocular surface a therapeutically effective amount of a preparation comprising a composition comprising a compound selected from the group consisting of galectin-1, galectin-3, PDGF, bFGF, or functional fragments thereof, wherein the preparation for administration further comprises dispersion in a carrier vehicle selected from drops of liquid, gels, ointments, or liposomes.

A contact lens comprising a composition comprising a compound selected from the group consisting of galectin-1, galectin-3, PDGF, bFGF, or functional fragments thereof.

### BRIEF DESCRIPTION OF DRAWINGS

FIG. 1 is a graph showing the increase cell proliferation of rat conjunctival goblet cells as a function of contact with increasing amounts of galectin 1 or 3.

FIG. 2 is a graph showing the increase cell proliferation of rat conjunctival goblet cells as a function of contact with increasing amounts of PDGF or bFGF . Other growth factors were also tested.

### DETAILED DESCRIPTION

The present invention provides methods for regulating goblet cells and their secretions comprising contacting goblet cells with an effective amount of a composition to increase or decrease goblet cell number and/or secretions. In some embodiments, the composition comprises a galectin, basic fibroblast growth factor (bFGF), platelet derived growth factor (PDGF), or combinations thereof. In some embodiments, the composition comprises galectin-1 and/or galectin-3. In some embodiments, the composition is an ophthalmic composition.

As used herein, the term “ophthalmic composition” refers to a composition suitable for administration to the eye or ocular surface. The composition can be in any form as described herein and equivalents thereof.

The compositions of the present invention can comprise, basic fibroblast growth factor (bFGF, also referred to as FGF-2), which is a growth factor that has been previously identified to be involved in cell proliferation. bFGF has several domains including, but not limited to, a kinase domain and a heparin binding domain.

The compositions of the present invention can comprise PDGF, which is a receptor that dimerizes and is autophosphorylated upon binding of its ligand. PDGF has several domains including, but not limited to, a kinase domain and a PDZ domain. PDGF is involved in cell proliferation. A “PDZ domain” is a modular protein interaction domain that binds in a sequence-specific fashion to short C-terminal peptides or internal peptides that fold in a beta-finger. (Sheng and Sala. Annu Rev Neurosci. (2001);24:1-29). In some embodiments, the composition comprises a domain of PDGF, including but not limited to, a PDZ domain.

The compositions of the present invention can comprise galectins, which are a family of proteins that contain a carbohydrate-binding domain (CRD) that has a conserved consensus region and galactose-specific lectin activity. Galectin-1, -2, -5, -7, -10, -11, -13, and -14 contain a single CRD domain (“mono-CRD galectins”). Galectin-4, -6, -8, -9, and -10 contain two CRD domains interconnected by an unconserved linker region (“bi-CRD galectins”). Galectin-3 has a chimeric structure consisting of a single CRD domain and an extended N-terminal region. The mono-CRD galectins can occur as monomers, dimers, or higher order oligomers and can be homo- or heteromers. The bi-CRD galectins are bivalent monomers (two binding sites) or oligomers. The galectins can be produced recombinantly using available techniques and information. For example, the nucleotide and protein sequences for galectin-1 and galectin-3, see, NM_{—}002305 and NM_{—}002306, respectively. Galectins are also reviewed in, for example, Glycoconjugate, Vol. 19, pages 443-629, which is hereby incorporated by reference in its entirety Galectins can also be purchased from Cell Services, Massachusetts.

The galectins of the present invention are synthesized in the cytosol on cytosolic ribosomes, but have no signal peptides and thus are not secreted by classical mechanisms. Galectins can, however, be phosphorylated. Once synthesized in the cytosol, galectins can be targeted to the nucleus or other subcellular sites, or they can also be secreted by non-endoplasmic reticulum/Golgi pathways. Thus, galectins function both intracellularly and extracellularly. Secretion of galectins can result in extracellular functions such as cell activation by autocrine or paracrine mechanisms and mediation of cell interaction or adhesion to the extracellular matrix.

Galectins are often multifunctional and multicompartmented and are regulated by various cellular factors. For example, when exogenously added to cells, galectin-1 can either promote or inhibit cell growth, depending upon the cell type. Galectin-1 stimulates proliferation of vascular endothelial cells, 3T3 fibroblasts, and corneal epithelial cells and induces regeneration of axons. Galectin-1, however, can inhibit proliferation of neuroblastoma cells, embryonic fibroblasts, mononuclear cells, T lymphocytes, and T lymphoma cells.

Similarly, exogenously added galectin-3 can either stimulate or inhibit cell proliferation. Extracellular galectin-3 stimulates the growth of fibroblasts, mesangial cells, and neurite outgrowth from dorsal root ganglia explants. In contrast, galectin-3 can inhibit growth of MDCK cells and bone marrow cells. Galectins can also cause cell apoptosis and can inhibit cell adhesion.

The effects of galectins at high concentration are, generally, carbohydrate dependent, but at low concentration can be carbohydrate independent. The carbohydrate dependent effects are potentially mediated by ganglioside GM 1.

In some embodiments, the present invention provides pharmaceutical compositions comprising a galectin, bFGF, PDGF, or combinations thereof. In some embodiments, the pharmaceutical composition is an ophthalmic composition. In some embodiments, the composition comprises galectin-1 and/or galectin-3. In some embodiments, the composition comprises a functional fragment of a galectin. In some embodiments, the composition comprises a therapeutically effective amount of galectin-1 or functional fragments thereof and/or galectin-3 or functional fragments thereof or a therapeutically effective amount of bFGF or functional fragments thereof, PDGF or functional fragments thereof, or combinations thereof. In some embodiments, the composition comprising an amount of a galectin (e.g. galectin-1 or galectin-3), bFGF, PDGF, or a combination thereof is effective to increase goblet cell proliferation and/or secretion.

In some embodiments, the methods of the present invention comprise contacting goblet cells with a composition comprising galectin-1, galectin-3, bFGF, PDGF, or a combination thereof to increase goblet cell number. In some embodiments, the composition comprises fragments of bFGF, PDGF, galectin-1, galectin-3, or combinations thereof. In some embodiments the goblet cell is a conjunctival goblet cell.

The present invention also provides methods of increasing goblet cell secretion comprising contacting goblet cells with a galectin or functional fragments thereof, bFGF, PDGF, or a combination thereof. In some embodiments, the galectin is galectin-1 or galectin-3. In other embodiments, a combination of galectins or functional fragments thereof, bFGF, PDGF, or a combination thereof are contacted with a goblet cell to increase goblet cell secretions. In some embodiments functional fragments of bFGF or PDGF are contacted with a goblet cell to increase goblet cell secretions.

A “goblet cell secretion” can be any secretion that can occur from a goblet cell. In some embodiments it is mucous (e.g. mucin) secretion.

The present invention also provides for methods of treating dry eye in an individual comprising contacting an ocular surface of the individual with a composition comprising a galectin or functional fragment thereof, bFGF or a functional fragment thereof, PDGF or a functional fragment thereof, or a combination thereof. In some embodiments, the galectin is galectin-1 or galectin-3. In some embodiments, the individual is an individual in need of such treatment.

As used herein, an individual in need thereof is an individual that has been identified as being diagnosed with a condition or disorder to be treated with the compositions and methods described herein.

In some embodiments, the present invention provides methods of increasing eye comfort during contact lens use comprising a topical administration of a composition comprising a therapeutically effective amount of a galectin, bFGF, PDGF, or a combination thereof contacted with an ocular surface. In some embodiments, functional fragments of a galectin, bFGF, or PDGF, or a combination thereof are contacted with an ocular surface. As discussed below, the composition can be contacted with an ocular surface by any means including, but not limited to, in the form of a topical ophthalmic composition.

The present invention also provides methods of lubricating an ocular surface of a contact lens wearer comprising contacting an ocular surface with a topical administration of a composition comprising a galectin or functional fragment thereof, bFGF or functional fragment thereof, PDGF or functional fragment thereof, or a combination thereof.

In some embodiments, the present invention provides sterile preparations adapted for topical administration to the eye comprising an amount of galectin-1 and/or galectin-3 or functional fragments thereof, bFGF or a functional fragment thereof, PDGF a functional fragment thereof, or a combination thereof. In some embodiments, the sterile preparation comprises an amount of a galectin, bFGF, PDGF, or a combination thereof effective to increase goblet cell proliferation and/or secretion.

The present invention also provides methods of stimulating goblet cell mucous secretion comprising contacting an ocular surface with a topical administration comprising a therapeutically effective amount of a preparation comprising a galectin, bFGF or a functional fragment thereof, PDGF or a functional fragment thereof, or a combination thereof. In some embodiments, the galectin is galectin-1 and/or galectin-3 or functional fragments thereof.

The present invention provides methods of stimulating goblet cell proliferation comprising contacting an ocular surface with a topical administration comprising a therapeutically effective amount of a preparation comprising a galectin, bFGF or a functional fragment thereof, PDGF or a functional fragment thereof, or a combination thereof. In some embodiments, the galectin is galectin-1 and/or galectin-3 or functional fragments thereof.

In some embodiments, the present invention provides method of identifying a functional fragment of a galectin, bFGF, or PDGF for increasing goblet cell proliferation or increasing goblet cell secretions comprising contacting a fragment of a galectin, bFGF, or PDGF with a goblet cell and measuring goblet cell proliferation and/or secretions, wherein an increase in proliferation or secretions indicates that said fragment is a functional fragment. In some embodiments, the secretion is mucin, electrolytes, and/or water.

As used herein, the phrase “functional fragment” refers to a fragment of a galectin, bFGF, or PDGF that is sufficient to either increase goblet cell proliferation or increase goblet cell secretion of mucin. In some embodiments, the functional fragment of a galectin comprises the carbohydrate binding domain of a galectin. Functional fragments can be readily ascertained using the assays described in the examples below. In some embodiments of the present invention functional fragments of bFGF or PDGF can be used. In some embodiments, the functional fragment of bFGF comprises the heparin binding domain. In some embodiments, the functional fragment of PDGF comprises the PDZ domain.

Any means can be used to measure goblet cell proliferation and/or goblet cell secretions. One of ordinary skill in the art knows how to measure cell proliferation and/or goblet cell secretions. Examples for measuring goblet cell proliferation and/or secretion are described in detail below. The culturing of goblet cells is known to one of skill in the art and is described in, for example, in U.S. Pat. No. 5,545,617, which is hereby incorporated by reference in its entirety.

For example, cell proliferation can be measured by counting the number of cells or using a marker of cell proliferation, such as but not limited to, water soluble tetrazolium salts (WST). One such method of measuring goblet cell secretion and/or proliferation can be found in U.S. Pat. No. 5,545,617, however, any method can be used.

Cell proliferation can also be measured by using an antibody to of Ki-67, which is a marker of cells in S-phase. Ki-67 is visualized using a fluorescently labeled secondary antibody.

To determine if the proliferating cells are goblet cells, cells are stained with Ulex Europeus Agglutinin I (“UEA-1”, a lectin) conjugated to a fluorophore. DAPi is a fluorescent molecule that is included in the mounting medium and labels the nuclei of all cells. Each fluorophore used fluoresces at a different wavelength. The number of cells that contain Ki-67 staining in the nucleus are counted using a fluorescence microscope. The number of Ki-67 positive cells that contain UEA-1 fluorescence is counted. This indicates the number of proliferating goblet cells. The number of DAPi positive cells are counted. This indicates the total number of cells. The number of proliferating goblet cells is expressed as a percentage of total number of cells,

Other methods for culturing goblet cells and measuring secretions, proliferation, and the like can be found in, for example, Dartt et al, Experimental Eye Research, (1996), 63:27-34; Horikawa et al., Investigative Ophthalmology & Visual Science, (2003), 44:2535-2544; Dartt et al., Experimental Eye Research (2000) 71:619-628; Kanno et al., American Journal of Cell Physiology 284:C988-C998; Dartt et al., Current Eye Research (1995) 14:993-1000; Kessler et al., Current Eye Research (1995) 14:985-992; Shatos et al., Investigative Ophthalmology & Visual Science, (2001) 42:1455-1464; or Shatos et al., Investigative Ophthalmology & Visual Science, (2003) 43:2477-2485, each of which is incorporated by reference in its entirety.

As a general non-limiting example, a galectin, bFGF, PDGF, or a combination thereof or any composition comprising a galectin, bFGF, PDGF, or a combination thereof can be contacted with goblet cells or a tissue comprising goblet cells. The proliferation of the goblet cells can be measured directly by staining the tissue for goblet cells or by isolating the goblet cells and counting the number and determining if the number of goblet cells increased, decreased, or remained constant in response to being contacted with a composition (e.g. a galectin). Other methods for measuring cell proliferation that can be used are described herein. In some embodiments, a functional fragment is used.

In some embodiments, the cell secretion that is measured is mucin secretion. In some embodiments, the present invention is directed to methods of modulating (e.g. increasing or decreasing) mucin production in eyes for any reason, including, but not limited to, treatment of a dry eye disease and/or condition. In some embodiments, the method comprises contacting an eye with a composition comprising a galectin (e.g. galectin-1 and/or galectin-3 or functional fragments thereof), bFGF or a functional fragment thereof, PDGF or a functional fragment thereof, or a combination thereof.

As used herein “dry eye” diseases or disorders are defined to include, but are not limited to: dry eye due to refractive surgery (corrective laser surgery), keratoconjunctivitis sicca (KCS), age-related dry eye, Stevens-Johnson syndrome, Sjogren's syndrome, ocular cicatrical pemphigoid, blepharitis, corneal injury, infection, Riley-Day syndrome, congenital alacrima, nutritional disorders or deficiencies (including vitamin), pharmacologic side effects, eye stress, glandular and tissue destruction, environmental exposure (e.g. smog, smoke, excessively dry air, airborne particulates), contact lens related dry eye, autoimmune and other immunodeficient disorders, and comatose patients rendered unable to blink. Dry eye can also be defined as a condition with a decrease or change in quality of tears irrespective of the presence or absence of corneal and conjunctival lesion. It includes dry eye conditions found in the patients of hypolacrimation, alacrima, xerophthalmia, and diabetes, etc.; post-cataract surgery dry eye; allergic conjunctivitis-associated dry eye; and age-related dry-eye syndrome. Dry eye can also include the conditions found in hypolacrimation patients induced by long time visual display terminal (VDT) operations, room dryness due to air-conditioning, and the like. Contact lens related dry eye (“CLRDE”) is a disorder marked by at least one objective clinical symptom and at least one subjective symptom. Clinical symptoms are selected from (a) a tear film break up time (“TFBUT”) of less than about 10 seconds in at least one eye; (b) a fluorescein staining score ≧3 on a scale of 0-15 in at least one eye; (c) a lissamine green staining score ≧3 on a scale of 0-18 in at least one eye; or (d) a tear meniscus grade of ‘abnormal’ in at least one eye. Subjective symptoms determined via patient feedback and include (a) ≧2 hour difference between average daily contact lens wear time and average daily comfortable contact lens wear time and (b) a rating of frequent or constant feelings of dryness, burning, stinging or discomfort during lens wear.

The present invention may also be useful to treat eye discomfort as a result of, for example, pollutants, allergies, strain, or discomfort may occur during surgery, or to maintain comatose patients or those who cannot blink due to muscle or nerve damage, neuromuscular blockade, or loss of the eyelids.

Topical administration of a composition according to the invention comprises infusion or instillation of the preparation, composition, or topical administration from a device selected from a group consisting of a pump-catheter system, a selective release device, and a contact lens. The preparation for topical administration can comprise dispersion of the preparation in a carrier vehicle selected from a group consisting of liquids, gels, ointments, and liposomes.

The present invention also provides contact lenses comprising a galectin or a functional fragment thereof, bFGF or a functional fragment thereof, PDGF or a functional fragment thereof, or a combination thereof. In some embodiments, the contact lens comprises galectin-1 , galectin-3, or both. Any ophthalmic device which resides on the eye may be used as a carrier for the compositions of the present invention. These devices can provide optical correction, wound care, drug delivery, diagnostic functionality, cosmetic enhancement or effect or a combination of these properties. The term lens includes but is not limited to soft contact lenses, hard contact lenses, overlay lenses, and optical inserts. Suitable contact lens can be made from any of a wide family of known materials including, but not limited to commercially available hydrogel formulations such as etafilcon, polymacon, vifilcon, genfilcon A, lenefilcon A, galyfilcon, senofilcon, omafilcon, balafilcon, lotrafilcon A, lotrafilcon B, comfilcon and the like. In other embodiments the lens formulations disclosed in U.S. Pat. No. 6,367,929, W003/022321, W002/022322, U.S. Pat. Nos. 6,846,892, 5,760,1000, 5,776,999, 5,789,461, 5,849,811, 5965,631, 6,867,245, 5,260,000, 5,070,215 and 5,610,252, 5,932,674, 4,978,713; 5,782,460, WO 98/42497, the disclosures of which are incorporated herein in their entireties. The compositions may be incorporated into or onto the contact lens by any method, such as soaking, coating, grafting, non-covalent association, imprinting combinations and the like.

A preparation or composition according to the invention can, by way of non-limiting illustration, be applied to the eye (e.g. ocular surface) in animals and humans as a drop or within ointments, gels, or liposomes. Further, the compounds may be infused or instilled into the tear film via a pump-catheter system. In other embodiments, the compounds can be contained within continuous or other selective-release devices, for example, but not limited to membranes. As a further example, the compounds can be attached to or carried by and/or contained within contact lenses that are placed on the eye. In general, it is desired that the mode of application be such that the compounds enter the tear film or make contact with the surface of the eye. In some embodiments, the composition or preparation can be contained within a swab or sponge which can be applied to an ocular surface. In some embodiments, of the present invention a composition or a preparation can be contained within a liquid spray which can be applied to the ocular surface. In some embodiments, a composition or preparation can be injected directly into the lacrimal tissues or onto the eye surface.

In some embodiments, the present invention provides methods of increasing the age at which individuals can wear a contact lens comprising administering composition to the ocular surface, wherein the composition comprises a galectin or a functional fragment thereof, bFGF or a functional fragment thereof, PDGF or a functional fragment thereof, or a combination thereof. In some embodiments, the administration is a topical administration.

In some embodiments a topical preparation is made by combining a composition (e.g. a galectin, bFGF, PDGF, functional fragments thereof, or a combination thereof) with an appropriate carrier and optionally preservative. The preparation typically can also contain a physiologically compatible vehicle, as those skilled in the art can select using conventional criteria. The vehicles can be selected from the known ophthalmic vehicles which include, but are not limited to, water, buffered aqueous solutions, polyethers such as polyethylene glycol, polyvinyls such as polyvinyl alcohol and povidone, cellulose derivatives such as methylcellulose and hydroxypropyl methylcellulose, petroleum derivatives such as mineral oil, white petrolatum, animal fats such as lanolin, vegetable fats such as peanut oil, polymers of acrylic acid such as carboxypolymethylene gel, polysaccharides such as dextrans, glycosaminoglycans such as sodium hyaluronate and salts such as sodium chloride and potassium chloride. In some embodiments the vehicle is any water-based solution that is useful for the packaging or storing of contact lenses. Typical solutions include, without limitation, saline solutions, other buffered solutions, and deionized water. Suitable saline solutions include salts including, without limitation, sodium chloride, sodium borate, sodium phosphate, sodium hydrogenphosphate, sodium dihydrogenphosphate, or the corresponding potassium salts of the same. These ingredients are generally combined to form buffered solutions that include an acid and its conjugate base, so that addition of acids and bases cause only a relatively small change in pH. The buffered solutions may additionally include 2-(N-morpholino)ethanesulfonic acid (MES), sodium hydroxide, 2,2-bis(hydroxymethyl)-2,2′,2″-nitrilotriethanol, n-tris(hydroxymethyl)methyl-2-aminoethanesulfonic acid, citric acid, sodium citrate, sodium carbonate, sodium bicarbonate, acetic acid, sodium acetate, ethylenediamine tetraacetic acid and the like and combinations thereof. In one embodiment, the solution is a borate buffered or phosphate buffered saline solution.

As used herein, the term “about” refers to a range of ±10% of the number that is being modified. For example, the phrase “about 10” would include both 9 and 11.

The articles “a”, “an”, and “the” are used herein to refer to one or to more than one (i.e., to at least one) of the grammatical object of the article. By way of example, “an element” means one element or more than one element.

In some embodiments the final osmolality or tonicity of the solution can vary. In some embodiments the preparation or composition can be diluted to hypotonic concentrations when this is therapeutically desirable. In some embodiments, the preparation or composition can also be concentrated to hypertonic concentrations when therapeutically desirable.

A “therapeutically effective amount” or an “effective amount” of a composition is any amount that is sufficient to provide the outcome desired. For example, a therapeutically effective amount or an effective amount of a composition to increase goblet cell proliferation could be different, but also could be the same, as a therapeutically effective amount or an effective amount to increase goblet cell secretion (e.g. mucin). One of skill in the art would readily be able to determine what is a therapeutically effective amount or an effective amount. A therapeutically effective amount can refer to an amount of a composition effective to prevent, alleviate or ameliorate symptoms of a disease, a disorder, or a condition in an individual.

In some embodiments, the compositions according to the present invention can be pharmaceutical preparations or ophthalmic preparations. Pharmaceutical compositions or preparations or ophthalmic compositions or preparations are formulated according to the mode of administration to be used. Compositions or preparations can include, for example, additives for isotonicity, which can include sodium chloride, dextrose, mannitol, sorbitol and lactose. In some cases, isotonic solutions such as phosphate or borate buffered saline are preferred. Stabilizers include gelatin and albumin. Alternatively the compositions may be dispersed to form an emulsion, such a liposome or double emulsions. The compositions and/or preparations according to the present invention can be sterile and pyrogen free. Pharmaceutical/Ophthalmic compositions according to the invention include delivery components in combination with the compositions for modulating goblet cell secretions or proliferation which further comprise pharmaceutically acceptable carriers or vehicles, such as, for example, saline, water, petroleum, dextran, combinations thereof and the like. Any medium can be used which allows for successful delivery of compositions for modulating goblet cell secretions or proliferation. One skilled in the art would readily comprehend the multitude of pharmaceutically acceptable media that can be used in the present invention. Suitable pharmaceutical carriers are described in Remington's Pharmaceutical Sciences, A. Osol, a standard reference text in this field, which is incorporated herein by reference.

The compositions according to the present invention can be administered as a single dose or in multiple doses. The pharmaceutical compositions of the present invention can be administered either as individual therapeutic agents or in combination with other therapeutic agents. The treatments of the present invention may be combined with conventional therapies, which can be administered sequentially or simultaneously.

Dosage varies depending upon known factors such as the pharmacodynamic characteristics of the particular agent, and its mode and route of administration; age; health and weight of the recipient; nature and extent of symptoms; kind of concurrent treatment; frequency of treatment; and the effect desired. Formulation of therapeutic compositions and their subsequent administration is within the skill of those in the art. The dosage range can be, for example, in about 1 ng/ml to about 1 mg/ml, about 2.5 ng/ml to about 30 ng/ml, about 10 ng/ml to about 25 ng/ml, about 0.1 μg/ml to about 10 μg/ml, about 1 μg/ml to about 8 μg/ml, about 2 μg /ml to about 5 μg /ml galectin, bFGF, PDGF or combination thereof/ml of preparation or composition. Doses can be, for example, about 2.5 ng/ml, about 5 ng/ml, about 10 ng/ml, about 15 ng/ml, about 20 ng/ml, about 25 ng/ml, about 0.1 μg /ml, about 0.5 μg/ml, about 1 μg/ml, about 2 μg/ml, about 3 μg/ml, about 4 μg/ml, or about 5 μg/ml galectin, bFGF, PDGF or combination thereof/ml of preparation or composition. The dosage range for the galectins will preferably be in the micromolar range; for bFGF and PDGF, the dosage range will preferably be in the nanomolar range. The concentrations described herein can refer to each active ingredient (e.g. galectin-1, galectin-3, bFGF, or PDGF) or to the total amount of active ingredients in a composition.

The present invention provides methods of identifying compounds for increasing mucin secretion. In some embodiments, the method comprises contacting a goblet cell with a first test compound determining if goblet cell proliferation is increased. If the goblet cell proliferation is increased this is indicative that the test compound increases mucin secretion. In some embodiments, the indication that the test compound increases mucin secretion is also indicative of the compound is able to be used to treat dry-eye conditions and other conditions affecting the eye as described herein. The test compound that is used in the present method or any method described herein can be any compound. In some embodiments, the test compound is a galectin, or a fragment thereof, bFGF or a fragment thereof, or PDGF or a fragment thereof. The galectin can be, for example, galectin-1 or galectin-3. The method can also comprise contacting the goblet cell with a second test compound. The second test compound can be contacted with the goblet cell prior to, simultaneously, or subsequent to contacting the goblet cell with the first test compound. Any number of test compounds can be tested at one time. The compounds can be contacted with a cell (e.g. goblet cell) serially (e.g. in any order) or at the same time. In some embodiments, two test compounds are contacted with a cell simultaneously and another test compound is added prior to or subsequent to the other compounds. In some embodiments, 2, 3, 4, 5, 6, 7, 8, 9, 10, or more than 10 compounds are tested on a goblet cell alone, serially, or in combination with one another.

One can also use the methods described herein to identify compounds that increase goblet cell proliferation.

The present invention also provides methods of identifying compounds for treating dry eye. The method can comprise contacting a goblet cell with a test compound and determining if goblet cell proliferation is increased. If an increase in goblet cell proliferation is observed or measured (quantitatively or qualitatively) this indicates that test compound could be used to treat dry eye. Additionally, goblet cell secretions could be observed or measured wherein an increase in goblet cell secretions indicates that the test compound could be used to treat dry eye. Any means of measuring goblet cell proliferation or secretions can be used. In some embodiments, a tetrazolium salt (WST) could be used to measure goblet cell proliferation. The secretion from a goblet cell can be any secreted compound including, but not limited to, mucin.

The method of identifying a test compound to treat dry eye, increase proliferation or cell secretion can be done either in vitro (e.g. cell culture) or in vivo (e.g. the test compound is contacted with an eye of an animal.

The invention is now described with reference to the following examples. These examples are provided for the purpose of illustration only and the invention should in no way be construed as being limited to these examples but rather should be construed to encompass any and all variations which become evident as a result of the teaching provided herein.

### EXAMPLES

### Example 1

Culture of Conjunctival Goblet Cells:

Conjunctival tissue from the fomical area and the nictitating membranes were removed from male Sprague Dawley rats. Tissue pieces were placed in explant culture in RPMI supplemented with fetal bovine serum (FBS). Over several days cells grew out of the explant and proliferated. Non-goblet cells, predominantly fibroblasts, were removed by scraping. Goblet cells were allowed to grow and proliferate for several days. Cells were removed from the tissue culture vessel by trypsin treatment. The number of cells was counted using a coulter counter. For measurement of proliferation 200 cells were seeded onto 96 well plates. Cells were allowed to attach and proliferate until a sparsely confluent level was obtained, about two days.

### Example 2

Optimizing Measurement of Goblet Cell Proliferation:

Before testing compounds, preparation and measurement techniques were optimized. Cells were cultured in increasing numbers in several wells and added WST (tetrazolium salts) to each well. It was found that increasing the number of cells increased the OD reading in a linear fashion. This finding demonstrates that use of WST in fact measures the number of cells and does so accurately.

Next, it was determined if the effect of the density of the starting culture altered the degree of stimulation of proliferation induced by FBS. Increasing numbers of cells from 200/well to 50,000/well were plated on 96 well plates. After serum starvation, 10% FBS was added. It was found that a minimum number (e.g. 200 to 500 per well of a 96 well plate) of cells per well were necessary to obtain stimulation of proliferation by FBS.

It was also investigated how many times goblet cells could be passaged and have them still proliferate in response to FBS. Passage 1 goblet cells were added to 96 well plates and the amount of cell proliferation in response to FBS determined after 1, 2, and 3 days of incubation. An aliquot of passage 1 cells was trypsinized and re-cultured as passage 2 cells. Proliferation was determined on these cells. The process was repeated on passage 3 cells. It was found that passage 1 and 2 cells proliferated in response to FBS, but passage 3 cells did not.

To ensure that the cells used were goblet cells, goblet cells were periodically cultured on glass cover slips. Cells were fixed in methanol and stained with the lectin UEA-I directly conjugated to the fluorophore rhodamine. Cells were visualized by immunofluorcesence microscopy. The overwhelming majority of cultured cells contained UEA-I positive immunoreactive staining in a punctate pattern in the cytoplasm indicating that they are goblet cells.

### Example 3

Measurement of Goblet Cell Proliferation:

A. Fluorescence Assay

Goblet cells from passage 1 or 2 cultured as above were used for measurement of proliferation. Cells were plated at 200 cells/well. Cells were serum-starved for 24 or 48 hrs. Test compounds in varying concentrations were added to each cell well for 24 hrs. The positive control for proliferation was the addition of 10% FBS, a known maximal stimulator of proliferation. Proliferation was terminated by the removal of medium. Cellular proliferation was measured using WST-1((2-(4-Iodophenyl)-3-(4-nitrophenyl)-5-(2,4-disulfophenyl)-2H-tetrazolium, sodium salt) and WST-8 (2-[2-methoxy-4- nitrophenyl]-3-[4-nitrophenyl]-5-[2,4-disulfophenyl]-2H- tetrazolium, monosodium salt), tetrazolium salts, that indicate the number of cell present based on the presence of active mitochondria. WST compound was added to each well and the amount of yellow color read using a fluorescence spectrophotometer at 465 nm. An increase in absorbance (OD) indicated an increase in cell number. Conditions were run in quintuplicate on cells from at least 3 different animals. The method demonstrated that the cells proliferated in response to FBS and could be measured by using WST.

B. Immunofluorescence Assay This assay was used to confirm the results from the fluorescence assay. Again, goblet cells from passage 1 or 2 were used. Equal numbers of cells (200) were plated onto glass cover slips. Cells were serum-starved for 24 hours. Media alone (basal), 10% FBS (positive control), or the concentration of test compound most effective in the fluorescence assay was added to the cover slips. After 24 hours, the coverslips were fixed in formaldehyde. After blocking in 4% bovine serum albumin and a permeabilizing agent, the primary antibody anti-Ki-67 was added.

Ki-67 was detected with a rhodamine secondary antibody using a filter that measures excitation wavelengths from 530-560 nm. UEA-1, which stains goblet cells, was directly conjugated to fluorescein isothyocyanate (FITC) and was detected using a filter that measures excitation wavelengths from 450-490 nm. DAPi, which stains the nuclei of all cells, was detected using a filter that measures excitation wavelengths from 400-418 nm. The percentage of proliferating goblet cells is determined by dividing the number of Ki-67 positive goblet cells by the total number of cells and multiplying by 100. Using this method, addition of FBS stimulated goblet cell proliferation compared to basal conditions.

### Example 4

Measurement of Goblet Cell Secretion:

Goblet cells from passage 1 or 2 as cultured above are used for measurement of cell secretion. Cells (500/well) are grown on 24-well plates to confluence (about 7 days). After serum starvation for 1 hour, compounds are added to each well for 1-8 hours. Medium is removed and analyzed for secreted, high-weight glycoproteins using an enzyme linked lectin assay with the lectin UEA-I. The assay is standardized using commercially available porcine stomach mucin. Cells are removed from each well and the amount of cellular protein is determined by a Bio Rad assay. The amount of protein is used to standardize the amount of secretion.

### Example 5

Compounds that Stimulate Goblet Cell Proliferation

Various compounds were tested for their effect on goblet cell proliferation as described in Example 3A.

1. Second Messengers: It was examined if goblet cell proliferation could be stimulated by increasing the levels of so called second messengers, that is, the signaling components that neurotransmitters and select growth factors activate. Compounds that increase cellular cAMP levels were tested. cAMP levels can be increased by activating adenylyl cyclase (e.g. with forskolin), the enzyme that synthesizes cAMP; by adding permeable cAMP analogues (e.g. dibutyryl cAMP); and by inhibiting the cAMP phosphodiesterase (e.g. 1-isobutyl-3methylxanthine), the enzyme that breaks down cAMP. Forskolin was added at 10^{-4;} butyryl cAMP was added directly to the medium at from 10⁻⁶ to 10⁻³ M; and 1-isobutyl-3methylxanthine was added directly to the medium at from 10⁻⁶ to 10⁻³ M. The second messengers did not increase proliferation at the concentrations tested; the positive control, 10% FBS, increased proliferation about 3 fold (data not shown).

Compounds that activate protein kinase C (PKC) were also tested. Phorbol myristic acid (PMA) activates almost all isoforms of PKC. PMA (solubilized in less than 1% DMSO) at 10⁻⁸M did not stimulate cell proliferation compared to no additions (0). Another set of phorbol esters were used, the alpha and beta isoforms of phorbol dibutyrate, which were solublized in DMSO as above (PdBu). The β isoform is active in activating PKC, whereas the α isoform is not. Neither isoform increased proliferation except at high concentrations for these compounds, i.e., greater than 10⁻⁷ M. At these concentrations, both isoforms increased proliferation, indicating an effect not mediated by PKC. The positive control (10% FBS) in these experiments increased proliferation about 4-fold.

2. Neural Agonists: Two different compounds that mimic the activation of nerves, carbachol and isoproterenol, were tested. Carbachol, a parasympathomimetic agonist, did not increase cell proliferation at any concentration (10⁻⁶ to 10⁻³ M) used. The positive control (10% FBS) increased proliferation about 3-fold. Isoproterenol, a sympathomimetic agonist that activates β-adrenergic receptors (10⁻⁷ to 10⁻³ M) also did not increase proliferation but was likely due to its interference with the assay (data not shown).

3. Growth Factors: Different families of growth factors that activate specific receptors were tested. Members of the epidermal growth factor (EGF) family that activate different EGF receptor (ErbB) subtypes were tested. All of the EGF family of growth factors were used at 10⁻⁷ M. EGF and transforming growth factor (TGF)α that activate ErbB-1 receptors significantly stimulated goblet cell proliferation about two-fold compared to control (no additions). Heparin binding-EGF (HB-EGF) that binds to ErbB-1 and ErbB-3 receptors also significantly increased proliferation 2-fold. Heregulin (HR) that binds to ErbB-3 and -4 receptors stimulated proliferation more than 2-fold. The positive control (10% FBS) increased proliferation 5-fold.

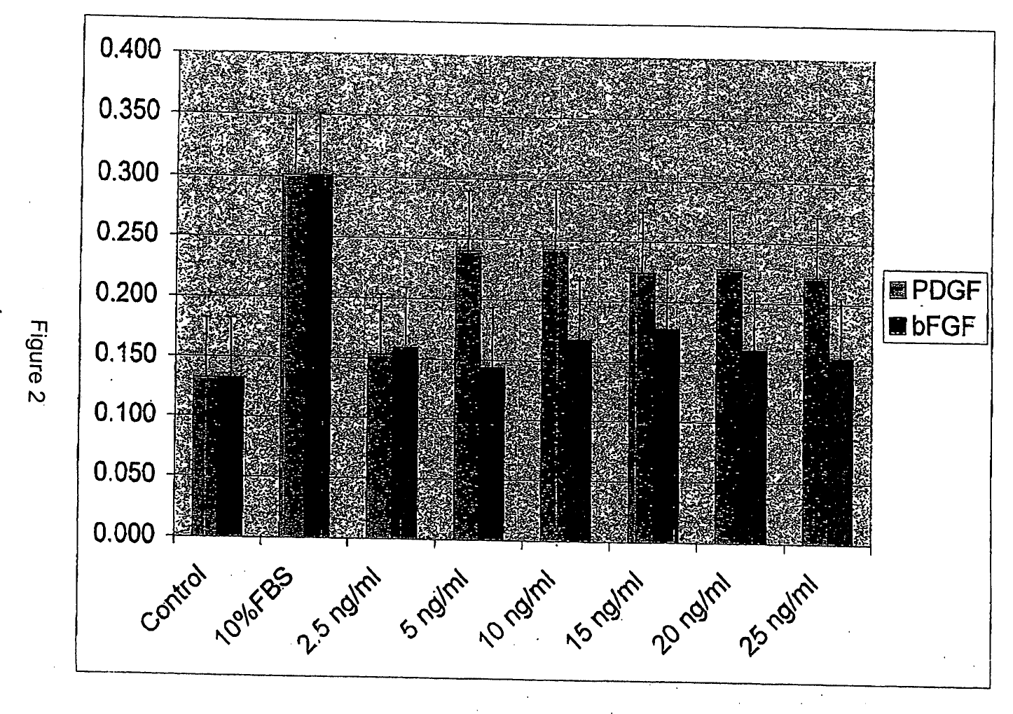

Platelet-derived growth factor (PDGF) was also tested. At concentrations from 5 to 25 ng/ml, PDGF increased proliferation 2-fold compared to no additions (0). The positive control increased proliferation about 2.5-fold (FIG. 2). Basic fibroblast growth factor (bFGF, at 10-20 ng/ml) also had similar effects (FIG. 2). Neither hepatocyte growth factor (HGF, 15-40 ng/ml) nor keratinocyte growth factor (KGF, 10-30 ng/ml), at any of the concentrations used, stimulated cell proliferation even though the positive control did. Bone morphogenic protein was also tested, but did not stimulate proliferation even at 100-300 ng/ml. Thyroid-stimulating hormone-alpha caused a small increase in proliferation at 10⁻⁷ to 10⁻⁸M.

The immunofluorescence assay (Example 3B) was used to confirm the above results with PDGF, bFGF, and EGF. Media alone gave no proliferation; 10% FBS caused 90% proliferation; bFGF caused 70% proliferation; and EGF caused 40% proliferation.

### Example 6

Galectin Stimulation of Goblet Cells

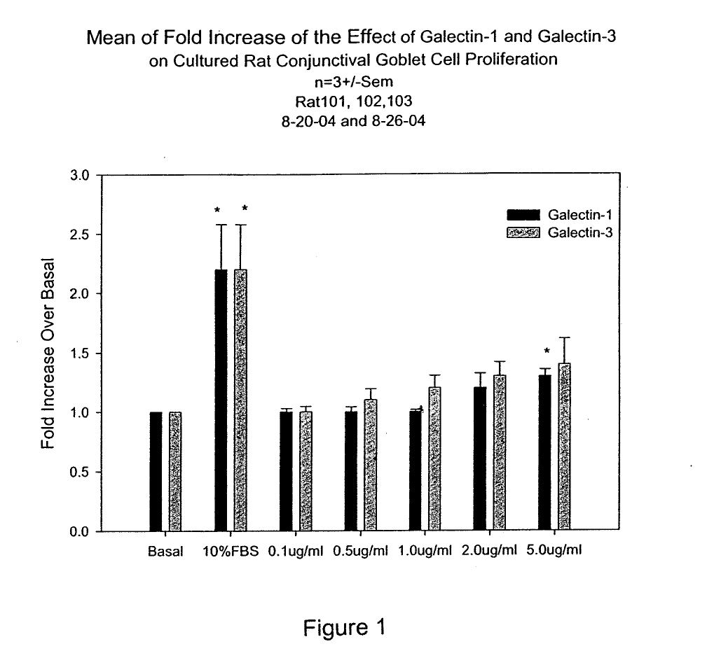

Galectin-1 or -3 (recombinant, obtained from Cell Services, Massachusetts, 98% pure) were tested for their effect on goblet cell proliferation as described in Example 3A above. Galectin-1 or -3 was added to goblet cells in increasing concentrations, ranging from 0.1 μg/ml to 5 μg/ml. The proliferation of the cells was measured. As shown in FIG. 1 and Table 1 below, both galectins stimulated goblet cell proliferation. The effect of galectin-1 or -3 on goblet cell proliferation appeared to be dose dependent (FIG. 1). As the amount of galectin was increased the amount of goblet cell proliferation also increased.

TABLE 1
Galectin-1

| | rat101 | rat102 | rat103 | Ave. fold | std | sem |
|---|---|---|---|---|---|---|
| Basal | 1.0 | 1.0 | 1.0 | 1.0 | 0.000 | 0.000 |
| 10% FBS | 2.9 | 2.3 | 1.5 | 2.2 | 0.657 | 0.379 |
| 0.1 ug/ml | 1.1 | 1.0 | 1.0 | 1.0 | 0.050 | 0.029 |
| 0.5 ug/ml | 1.1 | 1.0 | 1.0 | 1.0 | 0.074 | 0.043 |
| 1.0 ug/ml | 1.1 | 1.0 | 1.0 | 1.0 | 0.032 | 0.018 |
| 2.0 ug/ml | 1.5 | 1.2 | 1.1 | 1.2 | 0.213 | 0.123 |
| 5.0 ug/ml | 1.4 | 1.4 | 1.2 | 1.3 | 0.097 | 0.056 |

TABLE 2
Galectin-3

| | rat101 | rat102 | rat103 | Ave. fold | std | sem |
|---|---|---|---|---|---|---|
| Basal | 1.0 | 1.0 | 1.0 | 1.0 | 0 | 0 |
| 10% FBS | 2.9 | 2.3 | 1.5 | 2.2 | 0.657 | 0.379 |
| 0.1 ug/ml | 1.0 | 0.9 | 1.0 | 1.0 | 0.081 | 0.047 |
| 0.5 ug/ml | 1.3 | 1.0 | 1.0 | 1.1 | 0.160 | 0.092 |
| 1.0 ug/ml | 1.3 | 1.1 | 1.0 | 1.2 | 0.179 | 0.103 |
| 2.0 ug/ml | 1.5 | 1.3 | 1.1 | 1.3 | 0.202 | 0.117 |
| 5.0 ug/ml | 1.8 | 1.2 | 1.1 | 1.4 | 0.376 | 0.217 |

The disclosures of each and every patent, patent application, sequence information from accession numbers, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An ophthalmic composition comprising a therapeutically effective amount of galectin-1, galectin-3, or bFGF, or functional fragments thereof, to treat a dry eye disorder.

2. An ophthalmic composition comprising PDGF.

3. The ophthalmic composition of claim 2 wherein said composition comprises a therapeutically effective amount of PDGF to treat a dry eye disorder.

4. A pharmaceutical composition comprising a therapeutically effective amount of galectin-1, galectin-3, bFGF, or PDGF, or functional fragments thereof, to increase conjunctival goblet cell proliferation and/or secretion.

5. A method of increasing conjunctival goblet cell proliferation comprising contacting conjunctival goblet cells with a composition comprising galectin-1, galectin-3, bFGF, or PDGF, or functional fragments thereof.

6. The method of claim 5 wherein said composition is administered topically.

7. The method of claim 6 wherein said administration comprises infusion of said composition from a device selected from the group consisting of a pump-catheter system, a selective release device, and a contact lens.

8. The method of claim 6 comprising dispersion of said composition in a carrier vehicle selected from the group consisting of drops of liquid, gels, ointments, and liposomes.

9. A method of increasing conjunctival goblet cell secretion comprising contacting conjunctival goblet cells with a composition comprising galectin-1, galectin-3, bFGF, or PDGF, or functional fragments thereof.

10. The method of claim 9 wherein said secretion is mucous secretion.

11. The method of claim 9 wherein said composition is administered topically.

12. The method of claim 11, wherein said administration comprises infusion of said composition from a device selected from the group consisting of a pump-catheter system, a selective release device, and a contact lens.

13. The method of claim 11 comprising dispersion of said composition in a carrier vehicle selected from the group consisting of liquids, gels, ointments, and liposomes.

14. A method of treating dry eye in an individual comprising contacting an ocular surface of said individual with a composition comprising galectin-1, galectin-3, bFGF, or PDGF, or functional fragments thereof.

15. The method of claim 14 wherein said composition comprises eye drops.

16. A contact lens comprising a composition as in any one of claims 1, 2, or 4, or combinations thereof.
